SEQUENCE LISTING (8 sequences)

SEQ ID NO: 1 - protein, 260 aa, Homo sapiens
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 2 - protein, 260 aa, Artificial Sequence
  Description of Artificial Sequence Synthetic polypeptide
LKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 3 - protein, 305 aa, Artificial Sequence
  Description of Artificial Sequence Synthetic polypeptide
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 4 - protein, 299 aa, Artificial Sequence
  Description of Artificial Sequence Synthetic polypeptide
MGGPRALLAALWALEAAGTAALRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTGERAPPLPSRRALTPPPLPAAAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 5 - protein, 302 aa, Artificial Sequence
  Description of Artificial Sequence Synthetic polypeptide
MHYPTALLFLILANGAQAFRICAFNAQRLTLAKVAREQVMDTLVRILARCDIMVLQEVVDSSGSAIPLLLRELNRFDGSGPYSTLSSPQLGRSTYMETYVYFYRSHKTQVLSSYVYNDEDDVFAREPFVAQFSLPSNVLPSLVLVPLHTTPKAVEKELNALYDVFLEVSQHWQSKDVILLGDFNADCASLTKKRLDKLELRTEPGFHWVIADGEDTTVRASTHCTYDRVVLHGERCRSLLHTAAAFDFPTSFQLTEEEALNISDHYPVEVELKLSQAHSVQPLSLTVLLLLSLLSPQLCPAA

SEQ ID NO: 6 - protein, 22 aa, Artificial Sequence
  Description of Artificial Sequence Synthetic peptide
MRGMKLLGALLALAALLQGAVS

SEQ ID NO: 7 - protein, 20 aa, Artificial Sequence
  Description of Artificial Sequence Synthetic peptide
MYRMQLLSCIALSLALVTNS

SEQ ID NO: 8 - protein, 22 aa, Artificial Sequence
  Description of Artificial Sequence Synthetic peptide
MRYTGLMGTLLTLVNLLQLAGT

(19) United States
(12) Patent Application Publication
(10) Pub. No.: US 2024/0245758 A1
(43) Pub. Date: Jul. 25, 2024

(54) IMMUNOMODULATION OF TUMOR MICROENVIRONMENT

(71) Applicant: CLS THERAPEUTICS LIMITED, St. Peter Port, Guernsey (GG)

(72) Inventors: Dmitry Dmitrievich GENKIN, Saint Petersburg (RU); Georgy Viktorovich TETS, New York, NY (US); Viktor Veniaminovich TETS, New York, NY (US)

(73) Assignee: CLS THERAPEUTICS LIMITED, St. Peter Port, Guernsey (GG)

(21) Appl. No.: 18/563,348

(22) PCT Filed: May 20, 2022
(86) PCT No.: PCT/US2022/030168

§ 371 Date: Nov. 21, 2023

Related U.S. Application Data
(60) Provisional application No. 63/191,551, filed on May 21, 2021.

(51) Int. Cl. A61K 38/46; A61K 39/395; A61P 35/00

(52) U.S. Cl. CPC A61K 38/465; A61K 39/3955; A61P 35/00; C12Y 301/21001

(57) ABSTRACT

The invention relates to methods for immunomodulation of immunosuppressive tumor cell microenvironment and prevention of tumor microbiome effects through multiple pathways with DNase enzyme therapy. Methods for immunomodulation of immunosuppressive tumor cell microenvironment comprising administering an effective amount of deoxyribonuclease (DNase) enzyme, either alone or in combination with other immunomodulating agent.

### CROSS-REFERENCE TO RELATED APPLICATIONS

This application claims priority to U.S. Provisional Application No. 63/191,551, filed May 21, 2021, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on May 19, 2022, is named 252732_000038_SL.txt and is 14,097 bytes in size.

### FIELD OF THE INVENTION

The invention relates to new methods for immunomodulation of immunosuppressive tumor cell microenvironment and prevention of tumor microbiome effects in patients with different cancers utilizing a deoxyribonuclease (DNase) enzyme.

### BACKGROUND OF THE INVENTION

Patients with malignant tumors have elevated levels of circulating cell free DNA (cfDNA) in their plasma and serum as compared to healthy individuals (Fleischhacker, 2007). In cancer patients, circulating cfDNA originates both from dying non-tumor cells, tumor cells and neutrophils. Tumors predispose neutrophils to release extracellular DNA traps (NETs) that contribute to the establishment of a pro-thrombotic state, cachexia and organ failure in cancer patients (Demmers, 2012). Tumor originated cfDNA can contribute to the development of metastasis and chemotherapy resistance (Garcia-Olmo, 2013). The quantity of circulating cfDNA increases as the tumor progresses (Sawyers, 2008) reaching the maximal levels in patients with advanced disease and metastatic disease (Butt, 2008). It has been shown that higher quantities of circulating cfDNA significantly correlate with poor patient survival (Schwarzenbach, 2008).

The present inventors have previously demonstrated that systemic administration of high doses of DNase protein into a patient's circulation can be useful for treatment of a number of diseases and conditions associated with increased levels of cfDNA in the blood, including cancers (e.g., carcinomas, sarcomas, lymphomas, melanoma; see, e.g., U.S. Pat. Nos. 7,612,032; 8,710,012; 9,248,166), development of somatic mosaicism (see, e.g., U.S. Pat. Appl. Pub. No. US20170056482), and side effects associated with a chemotherapy or a radiation therapy (see, e.g., U.S. Pat. Appl. Pub. No. US20170100463). All of these patents and applications are incorporated by reference herein in their entireties. Others have later demonstrated similar effects. (Wen, 2013; Cederval, 2015; Tohme, 2016; Patutina, 2011; Li, 2015).

Immune checkpoint inhibitors (ICIs) are currently approved fora number of cancers. However, reported objective response rate to monotherapy range between 28-52% (Darvin et al., 2018).

### SUMMARY OF THE INVENTION

The present invention addresses great need in the art for new and more effective treatments of cancer, particularly those cancers which are not effectively treated with immune checkpoint modulators monotherapy.

In one aspect, the invention provides a method of immunomodulation of immunosuppressive tumor cell microenvironment in a subject having a cancer comprising administering to the subject an effective amount of a deoxyribonuclease (DNase) enzyme. In some embodiments, the immunomodulation comprises increased tumor cell killing by cytotoxic CD8 T cells and/or NK cells and/or CAR-T cells within immunosuppressive tumor cell microenvironment.

In another aspect, the invention provides a method of modulation of tumor-associated microbiome in a subject having a cancer comprising administering to the subject an effective amount of a deoxyribonuclease (DNase) enzyme.

In a further aspect, the invention provides a method of treating a cancer in a subject in need thereof, comprising administering to the subject an effective amount of a deoxyribonuclease (DNase) enzyme and a second immunomodulator.

In some embodiments of any of the above methods, the administration of the DNase enzyme is effective to reduce the number and/or activity of tumor associated macrophages (TAMs) and/or tumor infiltrating neutrophils (TINs) in immunosuppressive tumor cell microenvironment.

In some embodiments of the above methods involving administering a second immunomodulator, the second immunomodulator is an immune checkpoint modulator. In some embodiments, the immune checkpoint modulator is a modulator of an immune checkpoint molecule selected from PD-1, CD28, CTLA-4, CD137, CD40, CD134 (OX-40), ICOS, KIR, LAGS, CD27, TIM-3, BTLA, GITR, TCR, 4-1BB, TIGIT, CD96, CD226, KIR2DL, VISTA, HLLA2, TLIA, DNAM-1, CEACAMI, CD155, IDO, TGF-beta, IL-10, IL-2, IL-15, CSF-1, IL-6, adenosine A2A receptor (A2AR), and a ligand thereof. In some embodiments, the immune checkpoint modulator is an immune checkpoint inhibitor. In some embodiments, the immune checkpoint inhibitor is an antibody that specifically binds CTLA-4, PD-1, OX-40, PD-L1, or PD-L2. In some embodiments, the immune checkpoint inhibitor is an antibody that specifically binds to CTLA-4, PD-1, PD-L1, PD-L2, A2AR, B7-H3, B7-H4, BTLA, IDO, KIR, LAG 3, NOX2, TIM-3, VISTA, or SIGLEC7. In some embodiments, the immune checkpoint inhibitor is selected from ipilimumab, tremelimumab, nivolumab, pembrolizumab, pidilizumab, MEDI0680, atezolizumab, avelumab, durvalumab, cemiplimab, and any combinations thereof. In some embodiments, the immune checkpoint inhibitor is pembrolizumab.

In some embodiments of any of the above methods of the invention, the DNase enzyme is selected from human DNase I, human DNase-1-like 3 (DIL3), human DNase-1-like 2 (DIL2), human DNase-1-like 1 (DIL1), DNase X, DNase γ, DNase II, DNase IIα, DNase IIβ, and Caspase-activated DNase (CAD). In some embodiments, the DNase enzyme comprises an amino acid sequence having at least 90% sequence identity to human DNase I enzyme. In some embodiments, the DNase enzyme comprises an amino acid sequence having at least 90% sequence identity to SEQ ID NO: 1. In some embodiments, the DNase enzyme comprises an amino acid sequence having at least 90% sequence identity to amino acids 21 to 305 of DNase1-like 3 (DIL3) enzyme. In some embodiments, the DNase enzyme comprises an amino acid sequence having at least 90% sequence identity to amino acids 21 to 305 of SEQ ID NO: 3. In some embodiments, the DNase enzyme comprises the amino acid sequence SEQ ID NO: 2.

In some embodiments of any of the above methods of the invention, the DNase enzyme is administered as a DNase enzyme protein. In some embodiments, the DNase enzyme protein is administered intravenously for at least 2 days. In some embodiments, the DNase enzyme protein is administered intravenously for at least 7 days. In some embodiments, the DNase enzyme protein is administered intravenously for at least 14 days. In some embodiments, the DNase enzyme protein is administered intravenously for at least 16 days. In some embodiments, the DNase enzyme protein is administered intravenously from 1 to 2 days every 2 or 3 or 4 weeks, wherein the total length of treatment is from 2 weeks to 50 years. In some embodiments, the DNase enzyme protein is administered intravenously from 2 to 5 days every 2 or 3 or 4 weeks, wherein the total length of treatment is from 2 weeks to 50 years. In some embodiments, the DNase enzyme protein is administered intravenously from 7 to 14 days every 2 or 3 or 4 weeks, wherein the total length of treatment is from 2 weeks to 50 years. In some embodiments, the DNase enzyme protein is administered at 125-250 μg/kg/day.

In some embodiments of any of the methods involving administering of a second immunomodulator, the DNase enzyme protein is administered from 120 hours to 1 hour prior to administering the second immunomodulator. In some embodiments, the DNase enzyme protein is administered from 30 minutes to 2 hours after administering the second immunomodulator. In some embodiments, the DNase enzyme protein is administered from 2 hours to 360 hours after administering the second immunomodulator.

In some embodiments of any of the above methods of the invention, the DNase enzyme is encoded by a gene therapy vector. In some embodiments, said gene therapy vector is administered to the subject. In some embodiments, the gene therapy vector is a recombinant adeno-associated virus (rAAV) expression vector comprising (i) a capsid protein and (ii) a nucleic acid comprising a promoter operably linked to a nucleotide sequence encoding the DNase enzyme. In some embodiments, the promoter is selected from a liver-specific promoter, a nervous system-specific promoter, an intestine-specific promoter, a liver-specific/nervous system-specific tandem promoter, and a liver-specific/intestine-specific tandem promoter. In some embodiments, the promoter is specific for tumor originator tissue or metastasis target tissue. In some embodiments, the AAV is selected from serotype 1 (AAV1), AAV2, AAV3, AAV4, AAV5, AAV6, AAV7, AAV8, AAV9, AAV10, AAV11, AAV12, AAVrh10, AAVLK03, AAVLK06, AAVLK12, AAV-KP1, AAV-F, AAVDJ, AAVhu37, AAVrh64R1, and Anc 80.

In some embodiments of any of the above methods of the invention, the DNase enzyme is expressed by a cell comprising a chimeric antigen receptor (CAR) or a T cell receptor (TCR), and wherein said cell is administered to the subject. In some embodiments, the CAR expressing cell or the TCR expressing cell is administered directly to the site of the tumor. In some embodiments, the CAR expressing cell or TCR expressing cell is single-target or multi-target. In some embodiments, the CAR comprises an antigen binding domain capable of specific binding to one or more tumor antigens.

In some embodiments of any of the above methods of the invention involving the use of a second immunomodulator, the administration of the DNase enzyme is effective to reduce severity of one or more immune-related adverse events associated with the use of said second immunomodulator. In some embodiments, such immune-related adverse event is cytokine release syndrome (CRS). In some embodiments, the one or more immune-related adverse events are selected from uveitis, Sjögren syndrome, conjunctivitis, blepharitis, episcleritis, scleritis, retinitis, pneumonitis, pleuritis, sarcoid-like granulomatosis, hepatitis, pancreatitis, autoimmune diabetes, interstitial nephritis, glomerulonephritis, acute kidney injury (AKI), skin rash, pruritus, vitiligo, DRESS, psoriasis, Stevens-Johnson syndrome, arthralgia, arthritis, myositis, dermatomyositis, anaemia, neutropenia, thrombocytopenia, thrombotic microangiopathy, acquired haemophilia, vasculitis, colitis, enteritis, gastritis, myocarditis, pericarditis, hypophysitis, thyroiditis, adrenalitis, encephalitis, meningitis, polyneuropathy, Guillain-Barré syndrome, and subacute inflammatory neuropathies.

In some embodiments of any of the above methods of the invention, the administration of the DNase enzyme results in an alteration of the content and/or activity of tumor microbiome in the subject. In some embodiments, the tumor microbiome comprises one or more bacterial taxa selected from Acidobacteria, Actinobacteria, Bacteroidetes, Chlamydiae, Chrysiogenetes, Cyanobacteria, Fibrobacteres, Firmicutes, Fusobacteria, Gemmatimonadetes, Lentisphaerae, Proteobacteria, Spirochaetes, Synergistetes, Tenericutes, and Verrucomicrobia.

In some embodiments of any of the above methods of the invention, the DNase enzyme is administered prior to, together or after a cell therapy. In some embodiments, the cell therapy comprises administering (i) cells comprising a chimeric antigen receptor (CAR) or a T cell receptor (TCR), and/or (ii) NK cells, and/or (iii) CD8 T cells.

In some embodiments of any of the above methods of the invention, the subject is human.

In another aspect, the invention provides a pharmaceutical composition comprising a deoxyribonuclease (DNase) enzyme, a second immunomodulator, and a pharmaceutically acceptable carrier or excipient.

In a related aspect, the invention provides a pharmaceutical dosage form comprising a deoxyribonuclease (DNase) enzyme and a second immunomodulator.

In a further aspect, the invention provides a kit comprising a deoxyribonuclease (DNase) enzyme, a second immunomodulator, and optionally instructions for use.

In some embodiments of any of the above pharmaceutical compositions, dosage forms and kits, the second immunomodulator is an immune checkpoint modulator. In some embodiments, the immune checkpoint modulator is a modulator of an immune checkpoint molecule selected from PD-1, CD28, CTLA-4, CD137, CD40, CD134 (OX-40), ICOS, KIR, LAGS, CD27, TIM-3, BTLA, GITR, TCR, 4-1BB, TIGIT, CD96, CD226, KIR2DL, VISTA, HLLA2, TLIA, DNAM-1, CEACAMI, CD155, IDO, TGF-beta, IL-10, IL-2, IL-15, CSF-1, IL-6, adenosine A2A receptor (A2AR), and a ligand thereof. In some embodiments, the immune checkpoint modulator is an immune checkpoint inhibitor. In some embodiments, the immune checkpoint inhibitor is an antibody that specifically binds CTLA-4, PD-1, OX-40, PD-L1, or PD-L2. In some embodiments, the immune checkpoint inhibitor is an antibody that specifically binds to CTLA-4, PD-1, PD-L1, PD-L2, A2AR, B7-H3, B7-H4, BTLA, IDO, KIR, LAG 3, NOX2, TIM-3, VISTA, or SIGLEC7. In some embodiments, the immune checkpoint inhibitor is selected from ipilimumab, tremelimumab, nivolumab, pembrolizumab, pidilizumab, MEDI0680, atezolizumab, avelumab, durvalumab, cemiplimab, and any combinations thereof. In some embodiments, the immune checkpoint inhibitor is pembrolizumab.

In some embodiments of any of the above pharmaceutical compositions, dosage forms and kits, the DNase enzyme is selected from human DNase I, human DNase-1-like 3 (DIL3), human DNase-1-like 2 (DIL2), human DNase-1-like 1 (DIL1), DNase X, DNase γ, DNase II, DNase IIα, DNase IIβ, and Caspase-activated DNase (CAD). In some embodiments, the DNase enzyme comprises an amino acid sequence having at least 90% sequence identity to human DNase I enzyme. In some embodiments, the DNase enzyme comprises an amino acid sequence having at least 90% sequence identity to SEQ ID NO: 1. In some embodiments, the DNase enzyme comprises an amino acid sequence having at least 90% sequence identity to amino acids 21 to 305 of DNase1-like 3 (DIL3) enzyme. In some embodiments, the DNase enzyme comprises an amino acid sequence having at least 90% sequence identity to amino acids 21 to 305 of SEQ ID NO: 3. In some embodiments, the DNase enzyme comprises the amino acid sequence SEQ ID NO: 2.

In some embodiments of any of the above pharmaceutical compositions, dosage forms and kits, the DNase enzyme is present in the form of a DNase enzyme protein.

In some embodiments of any of the above pharmaceutical compositions, dosage forms and kits, the DNase enzyme is present in the form of a gene therapy vector encoding said DNase enzyme. In some embodiments, the gene therapy vector is a recombinant adeno-associated virus (rAAV) expression vector comprising (i) a capsid protein and (ii) a nucleic acid comprising a promoter operably linked to a nucleotide sequence encoding the DNase enzyme. In some embodiments, the promoter is selected from a liver-specific promoter, a nervous system-specific promoter, an intestine-specific promoter, a liver-specific/nervous system-specific tandem promoter, and a liver-specific/intestine-specific tandem promoter. In some embodiments, the promoter is specific for tumor originator tissue or metastasis target tissue. In some embodiments, the AAV is selected from serotype 1 (AAV1), AAV2, AAV3, AAV4, AAV5, AAV6, AAV7, AAV8, AAV9, AAV10, AAV11, AAV12, AAVrh10, AAVLK03, AAVLK06, AAVLK12, AAV-KP1, AAV-F, AAVDJ, AAVhu37, AAVrh64R1, and Anc 80.

In some embodiments of any of the above pharmaceutical compositions, dosage forms and kits, the DNase enzyme is present in the form of a cell which expresses said DNase and also comprises a chimeric antigen receptor (CAR) or a T cell receptor (TCR). In some embodiments, the CAR expressing cell or TCR expressing cell is single-target or multi-target. In some embodiments, the CAR comprises an antigen binding domain capable of specific binding to one or more tumor antigens.

These and other aspects described herein will be apparent to those of ordinary skill in the art in the following description, claims and drawings.

### BRIEF DESCRIPTION OF THE DRAWINGS

FIG. 1 Effect of DNase on (A) TAMs and (B) TINs on the models of mammary adenocarcinoma (MA), colorectal cancer (CRC), lung cancer (LC), pancreatic adenocarcinoma (PDAC). Amounts of TAMs and TIMs were normalized as the percentage compared with the average TAMs and TIMs in the PBS group (control, set as 100% CD45 in tumor) in each experiment.

FIG. 2 Effect of different therapeutic regimens of DNase administration on (A) TAMs and (B) TINs. Amounts of TAMs and TIMs were normalized as the percentage compared with the average TAMs and TIMs in the PBS group (control, set as 100% CD45 in tumor) in each experiment.

FIG. 3 Effect of DNase administration on (A) TAMs and (B) TINs, at different time periods post tumor implantation. Amounts of TAMs and TIMs were normalized as the percentage compared with the average TAMs and TIMs in the PBS group (control, set as 100% CD45 in tumor) in each experiment.

FIG. 4 Effect of DNase administration TAMs in combination with checkpoint inhibitors at different time periods post tumor implantation. Amounts of TAMs were normalized as the percentage compared with the average TAMs in the PBS group (control, set as 100% CD45 in tumor) in each experiment.

FIG. 5 Role of DNase on modulation of pro-tumorigenic effect of tumor microbiome. Tumor size was normalized as the percentage compared with the average size in the group 1 (control set as 100%) in each experiment.

FIG. 6 DNase prevents inhibition of checkpoint inhibitors activity by tumor microenvironment. Tumor size was normalized as the percentage compared with the average size in the group 1 (control set as 100%) in each experiment.

FIG. 7 DNase prevents toxicity associated with checkpoint inhibitor treatment. The graph shows body weights after administering test items to C57BL/6 mice bearing AT3 tumors. Data points represent group mean body weight. Error bars represent standard error of the mean (SEM). Administration of anti-CTLA4 antibody and F. nucleatum halts growth of body weight in treated animals. Combination of CTLA4 antibody and F. nucleatum leads to intensive weight loss. DNase I treatment prevents anti-CTLA4- and/or F. nucleatum-induced toxicity and rescues the weight gain.

### DETAILED DESCRIPTION OF THE INVENTION

The present invention is based on the inventors' hypothesis that targeting Neutrophil Extracellular Traps (NETs), an extracellular network of DNA and proteins expelled by neutrophils into the tumor microenvironment, may improve response rates to immune checkpoint therapy. Specifically, the present inventors decided to combine immune checkpoint therapy with a treatment with DNase, a NET depleting agent.

### Definitions

Unless defined otherwise, all technical and scientific terms used herein have the same meaning as commonly understood by one of ordinary skill in the art to which this invention belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “immunosuppressive tumor cell microenvironment” refers to the non-cancerous resident and infiltrating host cells, secreted factors and extracellular matrix proteins within tumor tissue collectively preventing tumor cell killing by cytotoxic CD8 T cells, NK cells and/or CAR-T cells.

The term “tumor-associated microbiome” refers to microorganisms, subcellular parts thereof and their metabolites localized within tumor.

The terms “extracellular DNA”, “cell-free DNA” and “cfDNA” are used interchangeably to refer to extracellular DNA (e.g., of eukaryotic, viral, archaeal, prokaryotic intracellular or extracellular parasites origin), including DNA in extracellular vesicles (e.g., exosomes and microvesicles), found in any bodily fluid and tissue. The cfDNA can be found in blood, lymph, liver, nervous tissues, cerebrospinal fluid (CSF), and/or intestine, including DNA in extracellular vesicles (e.g., exosomes and microvesicles) found in these bodily fluids and tissues.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The terms “individual”, “subject”, “animal”, “patient”, and “mammal” are used interchangeably to refer to mammals, including humans, veterinary animals (e.g., cats, dogs, cows, horses, sheep, pigs, etc.) and experimental animal models of diseases (e.g., mice, rats).

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a combination of DNase and another compound) the effective amount of the combination may or may not include amounts of each ingredient that would have been effective if administered individually.

As used herein, the term “promoter” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “tissue specific” promoter may be preferentially active in specific types of tissues or cells.

The term “liver-specific expression” as used herein refers to a predominant or exclusive expression in the liver, i.e., expression to a substantially greater extent than in other tissues and organs.

The term “liver-specific promoter” is used herein to refer to a promoter which is predominantly or exclusively active in a liver cell (e.g., hepatocyte) and directs/initiates transcription in the liver to a substantially greater extent than in other tissues and organs. In this context, the term “predominantly” means that at least 50% of said promoter-driven expression, more typically at least 90% of said promoter-driven expression (such as 100% of said promoter expression) occurs in liver cells. The ratio of liver expression to non-liver expression can vary between different liver-specific promoters. In some embodiments, a liver-specific promoter may preferentially direct/initiate transcription in a particular liver cell type (e.g., hepatocytes, Kupffer cells, endothelial cells, etc.). Some liver-specific promoters useful in the expression cassettes of the invention include at least one, typically several, hepatic nuclear factor binding sites. Liver-specific promoters useful in the expression cassettes of the invention can be constitutive or inducible promoters. Some non-limiting examples of hepatic promoters useful in the expression cassettes of the invention include: albumin promoter (Alb), human alpha-1 anti-trypsin (hAAT) promoter, thyroxine binding globulin (TBG), Apolipoprotein E hepatic control region promoter, Apolipoprotein A-II (APOA2) promoter, serpin peptidase inhibitor, clade A, member 1 (SERPINA1) (hAAT) promoter, cytochrome P450 family 3, subfamily A polypeptide 4 (CYP3A4) promoter, microRNA 122 (miR-122) promoter, liver-specific IGF-II promoter P1, murine transthyretin (MTTR) promoter, alpha-fetoprotein (AFP) promoter, lecithin-cholesterol acyl transferase (LCAT) promoter, apolipoprotein H (ApoH) promoter, and mouse prealbumin gene promoter.

Non-limiting examples of liver-specific promoters include, e.g., albumin promoter (Alb), human alpha-1 anti-trypsin (hAAT) promoter, thyroxine binding globulin (TBG) promoter, Apolipoprotein E hepatic control region promoter, Apolipoprotein A-II (APOA2) promoter, serpin peptidase inhibitor, clade A, member 1 (SERPINA1) (hAAT) promoter, cytochrome P450 family 3, subfamily A polypeptide 4 (CYP3A4) promoter, microRNA 122 (miR-122) promoter, Liver-specific IGF-II promoter P1, murine transthyretin (MTTR) promoter, the alpha-fetoprotein (AFP) promoter, a thyroid hormone-binding globulin promoter, an alcohol dehydrogenase promoter, the factor VIII (FVIII) promoter, a HBV basic core promoter (BCP) and PreS2 promoter, a phosphoenol pyruvate carboxykinase (PEPCK) promoter, an Hepatic Control Region (HCR)-ApoCII hybrid promoter, an AAT promoter combined with the mouse albumin gene enhancer (Ealb) element, a low density lipoprotein promoter, a pyruvate kinase promoter, a phosphenol pyruvate carboxykinase promoter, a lecithin-cholesterol acyl transferase (LCAT) promoter, an apolipoprotein H (ApoH) promoter, the transferrin promoter, a transthyretin promoter, an alpha-fibrinogen and beta-fibrinogen promoters, an alpha 1-antichymotrypsin promoter, an alpha 2-HS glycoprotein promoter, an haptoglobin promoter, a ceruloplasmin promoter, a plasminogen promoter, promoters of the complement proteins (e.g., Clq, Clr, C2, C3, C4, C5, C6, C8, C9, complement Factor I, and Factor H), C3 complement activator and the [alpha]1-acid glycoprotein promoter. Additional tissue-specific promoters may be found in the Tissue-Specific Promoter Database, TiProD (Nucleic Acids Research, J4:D104-D107 (2006).

The term “nervous system-specific promoter” is used herein to refer to a promoter which is predominantly or exclusively active in a nervous system cell and directs/initiates transcription in the nervous system (e.g., central nervous system (CNS), including brain, and/or enteric nervous system (ENS)) to a substantially greater extent than in other tissues and organs. In this context, the term “predominantly” means that at least 50% of said promoter-driven expression, more typically at least 90% of said promoter-driven expression (such as 100% of said promoter expression) occurs in cells of the nervous system. The ratio of nervous system expression to non-nervous system expression can vary between different nervous system-specific promoters. In some embodiments, a nervous system-specific promoter may preferentially direct/initiate transcription in a particular CNS and/or ENS cell type (e.g., neurons, glial cells [e.g., oligodendrocytes, astrocytes, ependymal cells, microglia, Schwann cells, satellite cells], enteric neurons, intrinsic primary afferent neurons, interneurons, motor neurons, etc.). Nervous system-specific promoters useful in the expression cassettes of the invention can be constitutive or inducible promoters. Some non-limiting examples of nervous system promoters useful in the expression cassettes of the invention include microglia-specific promoters (e.g., F4/80, CD68, TMEM119, CX3CR1, CMV, and Iba1 promoters), myeloid-specific promoters (e.g., TTR, CD11b, and c-fes promoters), neuron-specific promoters (e.g., CMV, NSE, synapsin [SynI, SynII], CamKII, α-CaMKII, and VGLUTI promoters), and other neural and glial cell (e.g., oligodendrocytes, astrocytes) type-specific promoters (e.g., glial fibrillary acidic protein [GFAP] promoter).

The term “intestine-specific promoter” is used herein to refer to a promoter which is predominantly or exclusively active in an intestinal cell and directs/initiates transcription in the intestine to a substantially greater extent than in other tissues and organs. In this context, the term “predominantly” means that at least 50% of said promoter-driven expression, more typically at least 90% of said promoter-driven expression (such as 100% of said promoter expression) occurs in intestine cells. The ratio of intestine expression to non-intestine expression can vary between different intestine-specific promoters. In some embodiments, an intestine-specific promoter may preferentially direct/initiate transcription in a particular intestinal cell type (e.g., enterocytes, goblet cells, enteroendocrine cells, etc.). Intestine-specific promoters useful in the expression cassettes of the invention can be constitutive or inducible promoters. Some non-limiting examples of intestine promoters useful in the expression cassettes of the invention include CB/CMV, GFAP, miCMV, CMV+I, tetO-CMV, β-acti-CMV, MUC2, Villin, and T3^{b} promoters.

In some embodiments, e.g., when tissue targeting is mediated by a viral capsid protein, a nucleic acid encoding a DNase enzyme can be operably linked to a promoter that allows for efficient systemic expression (e.g., CMV promoter, chicken β-actin promoter (CBA), or EF1a promoter).

The phrase “pharmaceutically acceptable”, as used in connection with compositions of the invention, refers to molecular entities and other ingredients of such compositions that are physiologically tolerable and do not typically produce untoward reactions when administered to a subject (e.g., a mammal such as a human). As used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the terms “viral vector” and “viral construct” refer to a recombinant viral construct that comprises one or more heterologous nucleotide sequences (e.g., a nucleotide sequence encoding a DNase enzyme). In some embodiments, the viral vector is replication deficient. In some embodiments, viral structural and non-structural coding sequences are not present in the viral vector and are provided during viral vector production in trans by a vector, such as a plasmid, or by stably integrating the sequences into a packaging cell line. Depending on the virus, a viral vector can be packaged within a capsid (e.g., an AAV vector) and/or a lipid envelope (e.g., a lentiviral vector).

In accordance with the present invention there may be employed conventional pharmacology and molecular biology techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985)); Transcription and Translation (B. D. Hames & S. J. Higgins, eds. (1984)); Animal Cell Culture (R. I. Freshney, ed. (1986); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994); among others.

### Methods of the Invention

In one aspect, the invention provides a method of immunomodulation of tumor microenvironment and/or prevention of tumor microbiome effects in a subject in need thereof, comprising administering to the subject a deoxyribonuclease (DNase) enzyme. In another aspect, the invention provides a method of reducing intensity of adverse events including immune-related adverse events of immune checkpoint modulator therapy via immunomodulation of tumor microenvironment and prevention of tumor microbiome effects.

In one aspect, the invention provides a method of immunomodulation of immunosuppressive tumor cell microenvironment in a subject having a cancer comprising administering to the subject an effective amount of a DNase enzyme.

In another aspect, the invention provides a method of modulation of tumor-associated microbiome in a subject having a cancer comprising administering to the subject an effective amount of a DNase enzyme.

In a further aspect, the invention provides a method of treating a cancer in a subject in need thereof, comprising administering to the subject an effective amount of a DNase enzyme and a second immunomodulator. In some embodiments, the second immunomodulator is an immune checkpoint modulator. In some embodiments, the immune checkpoint modulator is a modulator of an immune checkpoint molecule selected from PD-1, CD28, CTLA-4, CD137, CD40, CD134 (OX-40), ICOS, KIR, LAGS, CD27, TIM-3, BTLA, GITR, TCR, 4-1BB, TIGIT, CD96, CD226, KIR2DL, VISTA, HLLA2, TLIA, DNAM-1, CEACAMI, CD155, IDO, TGF-beta, IL-10, IL-2, IL-15, CSF-1, IL-6, adenosine A2A receptor (A2AR), and a ligand thereof. In some embodiments, the immune checkpoint modulator is an immune checkpoint inhibitor. Non-limiting examples of useful immune checkpoint inhibitors include, e.g., antibodies that specifically bind to CTLA-4, PD-1, PD-L1, PD-L2, OX-40, A2AR, B7-H3, B7-H4, BTLA, IDO, KIR, LAG 3, NOX2, TIM-3, VISTA, or SIGLEC7. In some embodiments, the immune checkpoint inhibitor is selected from ipilimumab, tremelimumab, nivolumab, pembrolizumab, pidilizumab, MEDI0680, atezolizumab, avelumab, durvalumab, cemiplimab, and any combinations thereof. In some embodiments, the administration of the DNase enzyme is effective to reduce severity of one or more immune-related adverse events associated with the use of the second immunomodulator. In some embodiments, the immune-related adverse event is cytokine release syndrome (CRS). Other non-limiting examples of immune-related adverse events include, e.g., uveitis, Sjögren syndrome, conjunctivitis, blepharitis, episcleritis, scleritis, retinitis, pneumonitis, pleuritis, sarcoid-like granulomatosis, hepatitis, pancreatitis, autoimmune diabetes, interstitial nephritis, glomerulonephritis, acute kidney injury (AKI), skin rash, pruritus, vitiligo, DRESS, psoriasis, Stevens-Johnson syndrome, arthralgia, arthritis, myositis, dermatomyositis, anaemia, neutropenia, thrombocytopenia, thrombotic microangiopathy, acquired haemophilia, vasculitis, colitis, enteritis, gastritis, myocarditis, pericarditis, hypophysitis, thyroiditis, adrenalitis, encephalitis, meningitis, polyneuropathy, Guillain-Barré syndrome, and subacute inflammatory neuropathies.

In some embodiments of any of the methods of the invention, the administration of the DNase enzyme results in an alteration of the content and/or activity of tumor microbiome in the subject. In some embodiments, the tumor microbiome comprises one or more bacterial taxa selected from Acidobacteria, Actinobacteria, Bacteroidetes, Chlamydiae, Chrysiogenetes, Cyanobacteria, Fibrobacteres, Firmicutes, Fusobacteria, Gemmatimonadetes, Lentisphaerae, Proteobacteria, Spirochaetes, Synergistetes, Tenericutes, and Verrucomicrobia.

In one aspect, the invention provides a method of immunomodulation of tumor microenvironment and prevention of tumor microbiome effects in a subject in need thereof, wherein the tumor microbiome is represented by bacteria, fungi, viruses.

In one aspect, aspect the invention provides a method of reducing intensity of adverse events including immune-related adverse events of immune checkpoint modulator therapy via immunomodulation of tumor microenvironment and prevention of tumor microbiome effects.

In one aspect, the invention provides a method of immunomodulation of tumor microenvironment and prevention of tumor microbiome effects in a subject in need thereof, wherein bacteria of tumor microbiome are from Acidobacteria, Actinobacteria, Bacteroidetes, Chlamydiae, Chrysiogenetes, Cyanobacteria, Fibrobacteres, Firmicutes, Fusobacteria, Gemmatimonadetes, Lentisphaerae, Proteobacteria, Spirochaetes, Synergistetes, Tenericutes, and/or Verrucomicrobia

In one aspect, the invention provides a method of immunomodulation of tumor microenvironment and prevention of tumor microbiome effects in a subject in need thereof, wherein bacteria of tumor microbiome are representatives of Fusobacteriales, Enterobacterales, and/or Bacillales.

In some embodiments of any of the above methods, the administration of the DNase enzyme is effective to reduce the number and/or activity of tumor associated macrophages (TAMs) and/or tumor infiltrating neutrophils (TINs) in immunosuppressive tumor cell microenvironment.

In some embodiments of any of the above methods, the DNase enzyme is selected from DNase I, DNase X, DNase γ, DNase1L1, DNase1L2, DNase 1L3, DNase II, DNase IIα, DNase IIβ, Caspase-activated DNase (CAD), Endonuclease G (ENDOG), Granzyme B (GZMB), and mutants or derivatives thereof.

In some embodiments of any of the above methods, the DNase enzyme is administered as DNase enzyme protein. In some embodiments, the DNase enzyme protein is administered parenterally.

In some embodiments of any of the above methods, the DNase enzyme is DNase I or a mutant or derivative thereof.

In some embodiments, the DNase I mutant comprises one or more mutations in an actin binding site. In some embodiments, the one or more mutations in the actin-binding site are selected from a mutation at Gln-9, Glu-13, Thr-14, His-44, Asp-53, Tyr-65, Val-66, Val-67, Glu-69, Asn-74, Ala-114, and any combinations thereof. In some embodiments, one of the mutations in the actin-binding site is a mutation at Ala-114. In some embodiments, the DNase I mutant comprises one or more mutations increasing DNase activity. In some embodiments, one or more mutations increasing DNase activity are selected from the group consisting of Q9R, E13R, E13K, T14R, T14K, H44R, H44K, N74K, A114F, and any combinations thereof. In some embodiments, one or more mutations increasing DNase activity are selected from the group consisting of Q9R, E13R, N74K and A114F, and any combi-nations thereof. In some embodiments, the DNase I mutant comprises the mutations Q9R, E13R, N74K, and A114F. In some embodiments, the DNase I mutant comprises one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N:T46S, D53R:Y65A, D53R:E69R, H44A:D53R:Y65A, H44A:Y65A:E69R, H64N: V66S, H64N: V66T, Y65N:V67S, Y65N:V67T, V66N:S68T, V67N:E69S, V67N:E69T, S68N:P70S, S68N:P70T, S94N:Y96S, S94N:Y96T, and any combinations thereof. In some embodiments, the DNase enzyme comprises an amino acid sequence having at least 90% sequence identity to human DNase I enzyme. In some embodiments, the DNase enzyme comprises an amino acid sequence having at least 90% sequence identity to amino acids 21 to 305 of DNase1-like 3 (DIL3) enzyme (SEQ ID NO:3).

In some embodiments of any of the above methods using a DNase enzyme protein, the DNase enzyme protein is injected intravenously for at least 14 days following infusion of the immune checkpoint modulator. In some embodiments, the DNase enzyme protein is injected intravenously for at least 16 days following infusion of the immune checkpoint modulator. In some embodiments, the DNase enzyme protein is injected intravenously for at least 2 days prior, together or following infusion of the immune checkpoint modulator. In some embodiments, the DNase enzyme protein is injected intravenously for at least 1 day prior or following infusion of the immune checkpoint modulator. In some embodiments, the DNase enzyme protein is injected intravenously for at least 7 days following infusion of the immune checkpoint modulator. In some embodiments, the DNase enzyme protein is injected intravenously for at least 7 days prior to infusion of immune checkpoint modulator. In some embodiments, the DNase enzyme protein is injected intravenously every other day for at least 2 days prior or following infusion of immune checkpoint modulator. In some embodiments, the DNase enzyme protein is injected intravenously every other day for at least 5 days prior or following infusion of immune checkpoint modulator. In some embodiments, the DNase enzyme protein is injected intravenously every other day for at least 7 days prior or following infusion of immune checkpoint modulator. In some embodiments, the DNase enzyme protein is injected intravenously every other day for at least 14 days prior or following infusion of immune checkpoint modulator. In some embodiments, the DNase enzyme protein is injected intravenously by intermittent course of total from 2 to 7 days prior or from 2 to 7 days following infusion of immune checkpoint modulator.

The administration of a DNase enzyme protein according to the methods of the invention can be performed by any suitable route, including systemic administration as well as administration directly to the site of the disease (e.g., to a primary tumor). Specific non-limiting examples of useful routes of administration include intravenous (IV), subcutaneous (SC), intraperitoneal (IP), oral, and intramuscular.

In certain embodiments, a DNase enzyme protein is formulated in a pharmaceutical composition with a pharmaceutically acceptable carrier or excipient.

In some embodiments of any of the above methods, the DNase enzyme is encoded by a gene therapy vector. In some embodiments, the gene therapy vector is administered to the subject. In some embodiments, the gene therapy vector is a viral vector. Non-limiting examples of useful viral vectors include, e.g., adeno-associated virus (AAV) vectors, adenoviral vectors, retroviral vectors (e.g., lentivirus vectors), and hepatotropic viral vectors (e.g., hepatitis B virus (HBV) vectors).

In some embodiments of any of the above methods using gene therapy vectors, the gene therapy vector is a recombinant adeno-associated virus (rAAV) expression vector comprising (i) a capsid protein and (ii) a nucleic acid comprising a promoter operably linked to a nucleotide sequence encoding a DNase enzyme. Non-limiting examples of AAV serotypes which can be used to develop the AAV expression vectors of the invention include, e.g., AAV serotype 1 (AAV1), AAV2, AAV3 (including types 3A and 3B), AAV4, AAV5, AAV6, AAV7, AAV8, AAV9, AAV10, AAV11, AAV12, AAVrh10, AAV-LK03, AAV-LK06, AAV-LK-01-19, AAV-LK12, AAV-KP1, AAVKP2-KP11, AAV-F, AAVrh64R1, AAVhu37, Anc80, Anc80L65, AAV-DJ, avian AAV, bovine AAV, canine AAV, equine AAV, ovine AAV, and chimeras thereof. In some embodiments, the promoter is specific for tumor originator tissue or metastasis target tissue. In some embodiments, the promoter is selected from a liver-specific promoter, a nervous system-specific promoter, an intestine-specific promoter, a liver-specific/nervous system-specific tandem promoter, and a liver-specific/intestine-specific tandem promoter.

In some embodiments of any of the above methods, the DNase enzyme is expressed by a cell comprising a chimeric antigen receptor (CAR) or a T cell receptor (TCR), and said cell is administered to the subject. In some embodiments, the CAR expressing cell or the TCR expressing cell is administered directly to the site of the tumor. In some embodiments, the CAR expressing cell or TCR expressing cell is single-target or multi-target. In some embodiments, the CAR comprises an antigen binding domain capable of specific binding to one or more tumor antigens. In some embodiments of any of the above methods, the CAR expressing cell or TCR expressing cell is further modified to express an immune checkpoint inhibitor molecule. In some embodiments, the CAR expressing cells are CAR T cells. In some embodiments, the CAR comprises an antigen binding domain capable of specific binding to one or more antigens selected from (i) a tumor antigen selected from CD5, CD7, CD19, CD28, mesothelin, CD123, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, IL-11Ra, PSCA, PRSS21, VEGFR2, LewisY, CD24, PDGFR-beta, SSEA-4, CD20, Folate receptor alpha, FR-1, c-MET, EGFR/CD133, IL13Ra2, HER2, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEMI/CD248, TEM7R, CLDN6, TSHR, GPRCSD, CXORF61, CD97, CD179a, ALK, Plysialic acid, PLAC1, GloboH, NY-BR-1, UPK2, HAVCRI, ADRB3, PANX3, GPR20, LY6K, OR51E2, TARP, WTI, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB1, BORIS, SART3, PAX5, 0Y-TES1, LCK, AKAP-4, SSX2, RAGE-1, human telomerase reverse transcriptase, RU1, RU2, intestinal carboxyl esterase, and mut hsp70-2; (ii) an antigen associated with a solid tumor; (iii) a solid tumor associated antigen selected from mesothelin, EGFRVIII, GD2, CLDN6, Tn Ag, PSMA, CD97, TAG72, CD44v6, CEA, EPCAM, KIT, IL-13Ra2, leguman, CD171, PSCA, TARP, MAD-CT-1, Lewis Y, CD24, folate receptor alpha, folate receptor beta, ERBBs, MUC1, EGFR, NCAM, PDGFR-beta, MAD-CT-2, Fos-related antigen, SSEA-4, neutrophil elastase, CAIX, HPV E6 E7, ML-IAP, NA17, ALK, androgen receptor plsialic acid, TRP-2, CYPIB1, PLAC1, GloboH, NY-BR-1, sperm protein 17, HMWMAA, beta human chorionic gonadotropin, AFP, thyroglobulin, RAGE-1, MN-CA IX, human telomerase reverse transcriptase, intestinal carboxyl esterase, and mut hsp 70-2; (iv) a solid tumor associated antigen present in/on a mesothelioma, a lung cancer, a pancreatic cancer, an esophageal adenocarcinoma, an ovarian cancer, a breast cancer, a colorectal cancer, a bladder cancer, or any combination thereof; (v) a tumor antigen that is associated with a hematological cancer; (vi) a tumor antigen present in a disease chosen from acute leukemias including B-cell acute lymphoid leukemia (“BALL”), T-cell acute lymphoid leukemia (“TALL”), and acute lymphoid leukemia (ALL); or one or more chronic leukemias including chronic myelogenous leukemia (CIVIL) and chronic lymphoid leukemia (CLL); and (vi) a tumor antigen present in a therapy resistant cancer.

### DNase Enzymes

As used herein, the terms “deoxyribonuclease” and “DNase” are used to refer to any enzyme that catalyzes the hydrolytic cleavage of phosphodiester linkages in the DNA backbone. A wide variety of deoxyribonucleases is known and can be used in the methods of the present invention. Non-limiting examples of DNases useful in the methods of the present invention include, e.g., DNase I (e.g., recombinant human DNase I (rhDNase I) or bovine pancreatic DNase I), analogues of DNase I (such as, e.g., DNase X, DNase γ, DNase1L1, DNase1L2, DNase 1L3), DNase II (e.g., DNase IIα, DNase IIβ), Caspase-activated DNase (CAD), Endonuclease G (ENDOG), Granzyme B (GZMB), phosphodiesterase I, lactoferrin, acetylcholinesterase, and mutants or derivatives thereof. Also encompassed by the present invention are DNase enzymes which have an extended half-life (e.g., albumin and/or Fc fusions, or protected from binding to actin by modification of actin binding-site; see, e.g., Gibson et al., (1992) J. Immunol. Methods, 155, 249-256). The actin binding site of DNase I can be mutated, for example, at the following residues: Gln-9, Glu-13, Thr-14, His-44, Asp-53, Tyr-65, Val-66, Val-67, Glu-69, Asn-74, Ala-114 of recombinant human DNase I (SEQ ID NO: 1). For example, one human DNase I hyperactive variant comprises mutated Ala-114 residue. Other exemplary mutations include, e.g., H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, H44N:T46S, D53R:Y65A, D53R:E69R, H44A:D53R: Y65A, H44A: Y65A:E69R, H64N: V66S, H64N:V66T, Y65N:V67S, Y65N:V67T, V66N:S68T, V67N:E69S, V67N:E69T, S68N:P70S, S68N:P70T, S94N:Y96S, S94N: Y96T (in the sequence of SEQ ID NO: 1). Also encompassed are mutations in DNase I with increased DNase I activity. Non-limiting examples of such mutations are, e.g., Q9R, E13R, E13K, T14R, T14K, H44R, H44K, N74K, and A114F of recombinant human DNase I (SEQ ID NO: 1). For example, one hyperactive DNase I mutant comprises a combination of the Q9R, E13R, N74K and A114F mutations. DNase I cleaves DNA preferentially at phosphodiester linkages adjacent to a pyrimidine nucleotide, yielding 5′-phosphate-terminated polynucleotides with a free hydroxyl group on position 3′, on average producing tetranucleotides. DNase I acts on single-stranded DNA, double-stranded DNA, and chromatin.

In some embodiments, the DNase may be a DNase I or a mutant or derivative thereof.

In some embodiments, the DNase I may be human DNase I or a mutant or derivative thereof. In some embodiments, the DNase I may be non-human DNase I or a mutant or derivative thereof, such as, but not limited to a rodent (e.g., a mouse) DNase I or a mutant or derivative thereof.

In some embodiments, the DNase enzyme comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to human DNase I enzyme. In some embodiments, the DNase enzyme comprises an amino acid sequence having at least 90%, sequence identity to human DNase I enzyme (SEQ ID NO: 1). In some embodiments, the DNase enzyme comprises an amino acid sequence having at least 90%, sequence identity to human DNase I enzyme mutant (SEQ ID NO: 2).

In some embodiments, the DNase enzyme comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to amino acids 21 to 305 of DNase1-like 3 (DIL3) enzyme. In some embodiments, the DNase enzyme comprises an amino acid sequence having at least 90% sequence identity to amino acids 21 to 305 of human DNase1-like 3 (DIL3) enzyme (SEQ ID NO: 3).

In some embodiments, the DNase enzyme comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to amino acids 21 to 305 of DNase1-like 2 (D1L2) enzyme. In some embodiments, the DNase enzyme comprises an amino acid sequence having at least 90% sequence identity to amino acids 21 to 305 of human DNase1-like 2 (D1L2) enzyme (SEQ ID NO: 4).

In some embodiments, the DNase enzyme comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to amino acids 21 to 305 of DNase1-like 1 (DIL1) enzyme. In some embodiments, the DNase enzyme comprises an amino acid sequence having at least 90% sequence identity to amino acids 21 to 305 of human DNase1-like 1 (DIL1) enzyme (SEQ ID NO: 5).

In some embodiments, the DNase is a DNase I mutant comprising one or more mutations in an actin binding site. In some embodiments, the one or more mutations in the actin-binding site are selected from a mutation at Gln-9, Glu-13, Thr-14, His-44, Asp-53, Tyr-65, Val-66, Val-67, Glu-69, Asn-74, Ala-114, and any combinations thereof. In some embodiments, one of the mutations in the actin-binding site is a mutation at Ala-114.

In some embodiments, the DNase is a DNase I mutant comprising one or more mutations increasing DNase activity. In some embodiments, one or more mutations increasing DNase activity are selected from the group consisting of Q9R, E13R, E13K, T14R, T14K, H44R, H44K, N74K, A114F, and any combinations thereof. In some embodiments, one or more mutations increasing DNase activity are selected from the group consisting of Q9R, E13R, N74K, and A114F.

In some embodiments, the DNase is a DNase I mutant comprising one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N:T46S, D53R:Y65A, D53R:E69R, H44A:D53R:Y65A, H44A:Y65A:E69R, H64N:V66S, H64N: V66T, Y65N:V67S, Y65N: V67T, V66N:S68T, V67N:E69S, V67N:E69T, S68N:P70S, S68N:P70T, S94N: Y96S, S94N:Y96T, and any combinations thereof. In some embodiments, the DNase I mutant is a long acting form of DNase. In some embodiments, the DNase I mutant is a hyperactive variant form of DNase. In some embodiments, the DNase I mutant comprises the amino acid sequence SEQ ID NO: 2.

In some embodiments, the DNaseI mutant comprises the mutations Q9R, E13R N74K and A114F.

In some embodiments, the DNaseI mutant comprises the mutations Q9R, E13R N74K and A114F.

In some embodiments of any of the methods of the invention, the sequence encoding the DNase comprises a secretory signal sequence. In some embodiments, where DNase is administered as a recombinant vector, said secretory signal sequence mediates effective secretion of the enzyme into the hepatic porto-sinusoidal circulation upon administration of the vector to the subject. In some embodiments, the secretory signal sequence is selected from the group consisting of DNase I secretory signal sequence, IL2 secretory signal sequence, albumin secretory signal sequence, ß-glucuronidase secretory signal sequence, alkaline protease secretory signal sequence, and fibronectin secretory signal sequence. In some embodiments, the secretory signal sequence comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence MRGMKLLGALLALAALLQGAVS (SEQ ID NO: 6). In some embodiments, the secretory signal sequence comprises the sequence MRGMKLLGALLALAALLQGAVS (SEQ ID NO: 6). In some embodiments, the secretory signal sequence consists of the sequence MRGMKLLGALLALAALLQGAVS (SEQ ID NO: 6). In some embodiments, the secretory signal sequence comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence MYRMQLLSCIALSLALVTNS (SEQ ID NO: 7). In some embodiments, the secretory signal sequence comprises the sequence MYRMQLLSCIALSLALVINS (SEQ ID NO: 7). In some embodiments, the secretory signal sequence consists of the sequence MYRMQLLSCIALSLALVTNS (SEQ ID NO: 7).

In some embodiments, the DNase enzyme is selected from the group consisting of DNase I, DNase X, DNase Y, DNase1L1, DNase1L2, DNase 1L3, DNase II, DNase IIα, DNase IIβ, Caspase-activated DNase (CAD), Endonuclease G (ENDOG), Granzyme B (GZMB), phosphodiesterase I, lactoferrin, acetylcholinesterase, and mutants or derivatives thereof. In some embodiments, the enzyme which has a DNase activity is DNase I or a mutant or derivative thereof. In some embodiments, the DNase I is a human DNase I or a mutant or derivative thereof. In some embodiments, the DNase I mutant comprises one or more mutations in an actin binding site. In some embodiments, the one or more mutations in the actin-binding site are selected from a mutation at Gln-9, Glu-13, Thr-14, His-44, Asp-53, Tyr-65, Val-66, Val-67, Glu-69, Asn-74, Ala-114, and any combinations thereof. In some embodiments, one of the mutations in the actin-binding site is a mutation at Ala-114. In some embodiments, the DNase I mutant comprises one or more mutations increasing DNase activity. In some embodiments, one or more mutations increasing DNase activity are selected from the group consisting of Q9R, E13R, E13K, T14R, T14K, H44R, H44K, N74K, A114F, and any combinations thereof. In some embodiments, one or more mutations increasing DNase activity are selected from the group consisting of Q9R, E13R, N74K and A114F, and any combinations thereof. In some embodiments, the DNase I mutant comprises a sequence having at least 80% or at least 85% or at least 90% or at least 95% sequence identity to the sequence of SEQ ID NO: 2. In some embodiments, the DNase I mutant comprises the mutations Q9R, E13R, N74K, and A114F. In some embodiments, the DNase I mutant comprises the sequence of SEQ ID NO: 2. In some embodiments, the DNAse I mutant consists of the sequence of SEQ ID NO: 2. In some embodiments, the DNase I mutant comprises one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N:T46S, D53R:Y65A, D53R:E69R, H44A:D53R: Y65A, H44A:Y65A:E69R, H64N:V66S, H64N:V66T, Y65N: V67S, Y65N:V67T, V66N:S68T, V67N:E69S, V67N:E69T, S68N:P70S, S68N:P70T, S94N:Y96S, S94N: Y96T, and any combinations thereof. In some embodiments, the enzyme which has a DNase activity is a fusion protein comprising (i) a DNase enzyme or a fragment thereof linked to (ii) an albumin or an Fc or a fragment thereof. In some embodiments, the sequence encoding the enzyme which has a DNase activity comprises a sequence encoding a secretory signal sequence, wherein said secretory signal sequence mediates effective secretion of the enzyme. In some embodiments, the secretory signal sequence is selected from the group consisting of DNase I secretory signal sequence, IL2 secretory signal sequence, the albumin secretory signal sequence, the β-glucuronidase secretory signal sequence, the alkaline protease secretory signal sequence, and the fibronectin secretory signal sequence. In some embodiments, the secretory signal sequence comprises the sequence MRGMKLLGALLALAALLQGAVS (SEQ ID NO: 6) or MYRMQLLSCIALSLALVINS (SEQ ID NO: 7). In some embodiments, the secretory signal sequence consists of the sequence MRGMKLLGALLALAALLQGAVS (SEQ ID NO: 6) or MYRMQLLSCIALSLALVTNS (SEQ ID NO: 7). In some embodiments, the secretory signal sequence comprises a sequence having at least 80% or at least 85% or at least 90% or at least 95% sequence identity to the sequence of MRGMKLLGALLALAALLQGAVS (SEQ ID NO: 6) or a sequence having at least 85% or at least 90% or at least 95% sequence identity to the sequence of MYRMQLLSCIALSLALVTNS (SEQ ID NO: 7). In some embodiments, the secretory signal sequence consists of the sequence MRYTGLMGTLLTLVNLLQLAGT (SEQ ID NO: 8). In some embodiments, the secretory signal sequence comprises a sequence having at least 80% or at least 85% or at least 90% or at least 95% sequence identity to the sequence of MRYTGLMGTLLTLVNLLQLAGT (SEQ ID NO: 8).

The administration of a DNase enzyme according to the methods of the invention can be performed by any suitable route, including systemic administration as well as administration directly to the site of the disease (e.g., to a primary tumor). Specific non-limiting examples of useful routes of administration include intravenous (IV), subcutaneous (SC), intraperitoneal (IP), oral, and intramuscular.

DNase enzyme protein doses useful in the methods of the invention depend on the type of additional therapy, the patient's clinical history and response to DNase, as well as the discretion of the attending physician. Non-limiting examples of useful dosage ranges include from 0.005 to 100 mg/kg/day or from 10 to 200000 KU/kg/day, preferably from 0.05 to 50 mg/kg/day or from 1000 to 100000 Kunitz units (KU)/kg/day, more preferably from 1.5 to 50 mg/kg/day or from 3000 to 100000 KU/kg/day, most preferably from 10 to 50 mg/kg/day or from 20000 to 100000 KU/kg/day.

In some embodiments, the DNase enzyme protein is injected intravenously at 250 μg/kg/day. In some embodiments, the deoxyribonuclease enzyme protein is injected intravenously for at least 14 days at 250 μg/kg/day.

### DNase Compositions and Formulations

In certain embodiments, a DNase enzyme protein, DNase enzyme-encoding vector or DNase enzyme expressing cell is formulated in a pharmaceutical composition with a pharmaceutically acceptable carrier or excipient. In certain embodiments, such composition further comprises a second immunomodulator.

The formulations used in the methods of the invention may conveniently be presented in unit dosage form and may be prepared by methods known in the art. The amount of active ingredients that can be combined with a carrier material to produce a single dosage form will vary depending upon the host being treated and the particular mode of administration. The amount of active ingredients that can be combined with a carrier material to produce a single dosage form will generally be that amount of the compound which produces a therapeutic effect.

In general, the formulations can be prepared with a liquid carrier, or a finely divided solid carrier, or both, and then, if necessary, shaping the product.

Pharmaceutical compositions suitable for parenteral administration may comprise one or more active ingredients ((i) a DNase enzyme protein, DNase enzyme-encoding vector or DNase enzyme expressing cell and, optionally, (ii) another compound [e.g., a second immunomodulator or another anti-cancer compound]) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms can be made by forming microencapsule matrices of one or more active ingredients in biodegradable polymers such as polylactide-polyglycolide. Depending on the ratio of active ingredient to polymer, and the nature of the particular polymer employed, the rate of active ingredient's release can be controlled. Examples of other biodegradable polymers include poly(orthoesters) and poly(anhydrides). Depot injectable formulations are also prepared by entrapping the active ingredients in liposomes or microemulsions which are compatible with body tissue.

Formulations for oral administration can be in the form of capsules, cachets, pills, tablets, powders, granules, or as a solution or a suspension in an aqueous or non-aqueous liquid (e.g., as a mouthwash, as a composition to be swallowed, or as an enema), or as an oil-in-water or water-in-oil liquid emulsion, and the like, each containing a predetermined amount of one or more active ingredients.

In solid dosage forms for oral administration (capsules, tablets, pills, dragees, powders, granules, and the like), one or more active ingredients can be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Suspensions, in addition to one or more active ingredients, can contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Powders and sprays can contain, in addition to one or more active ingredients, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

### Cancer and Other Treatments

The methods of the invention can be used in subjects suffering from a broad range of cancers. Non-limiting examples of relevant cancers include, e.g., breast cancer, prostate cancer, multiple myeloma, transitional cell carcinoma, lung cancer (e.g., non-small cell lung cancer (NSCLC)), renal cancer, thyroid cancer, leukemia (e.g., chronic myeloid leukemia, acute myeloid leukemia, chronic lymphocytic leukemia, acute lymphocytic leukemia), lymphoma (e.g., B cell lymphoma, T cell lymphoma, non-Hodgkin's lymphoma, Hodgkin's lymphoma), head and neck cancer, esophageal cancer, stomach cancer, colon cancer, intestinal cancer, colorectal cancer, rectal cancer, pancreatic cancer, liver cancer, cancer of the bile duct, cancer of the gall bladder, ovarian cancer, uterine endometrial cancer, vaginal cancer, cervical cancer, bladder cancer, neuroblastoma, sarcoma, osteosarcoma, malignant melanoma, squamous cell cancer, bone cancer, including both primary bone cancers (e.g., osteosarcoma, chondrosarcoma, Ewing's sarcoma, fibrosarcoma, malignant fibrous histiocytoma, adamantinoma, giant cell tumor, and chordoma) and secondary (metastatic) bone cancers, soft tissue sarcoma, basal cell carcinoma, angiosarcoma, hemangiosarcoma, myxosarcoma, liposarcoma, osteogenic sarcoma, angiosarcoma, endotheliosarcoma, lymphangiosarcoma, lymphangioendotheliosarcoma, synovioma, testicular cancer, uterine cancer, gastrointestinal cancer, mesothelioma, leiomyosarcoma, rhabdomyosarcoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, choriocarcinoma, seminoma, embryonal carcinoma, Wilms' tumor, epithelial carcinoma, glioma, glioblastoma, astrocytoma, medulloblastoma, craniopharyngioma, ependymoma, pinealoma, hemangioblastoma, acoustic neuroma, oligodendroglioma, meningioma, retinoblastoma, medullary carcinoma, thymoma, sarcoma, etc.

In some embodiments, the treatment methods of the invention can include, in addition to administering a DNase enzyme and a second immunomodulator, administering additional anti-cancer agents and/or therapies, including, without limitation, chemotherapeutic agents, radiation therapies, cell therapies (e.g., cells comprising a chimeric antigen receptor (CAR) or a T cell receptor (TCR), NK cells, CD8 T cells, etc.), and any combinations thereof.

In some embodiments of any of the methods of the invention, the subject is human.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described herein. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Deoxyribonuclease Enzyme Reduces the Number of Tumor-Associated Macrophages (TAMs) and Tumor Infiltrating Neutrophils (TINs) within Tumor Tissue

Based on the condition of the internal environment, circulating monocytes give rise to mature macrophages, and when they are recruited into the tumor microenvironment they are converted into tumor-associated macrophages (TAMs). TAMs not only lack the function of phagocytizing tumor cells but also help these tumor cells escape from being killed and help them spread to other tissues and organs. Tumor Infiltrating Neutrophils (TINs) are engaged into the tumor microenvironment by cytokines and chemokines. TANs stimulate immunosuppression, tumor growth, angiogenesis and metastasis by DNA instability, or by release of cytokines and/or chemokines.

The BALB/c mammary adenocarcinoma, MC38 colon carcinoma, Lewis lung carcinoma (LL2), and murine pancreatic adenocarcinoma (Panc2) cancer cell lines were used. 3×10e6 of mammary adenocarcinoma, 1×10e6 of MC38 cells, 3×10e5 of LL2 cells, or 2×10e5 Panc2 cells in 100 μl RPMI were administered by subcutaneous injection into mammary fat pad of syngeneic Balb/c or C57Bl/6 female mice that were from six to eight weeks of age.

Six days post tumor implantation, animals were randomly assigned to groups (N=3) and treated daily by IV injection of human recombinant DNase I at 75 μg (Kevelt AS; SEQ ID NO: 1) per mouse, control groups received the same amount of PBS. To isolate TAMs and TINs, tumors were extracted, and treated with 12 U/mL collagenase I (Sigma), 450 U/mL collagenase IV (Sigma), and 50 U/mL DNase I (Kevelt). Debris and dead cells were removed with density gradients. To purify TAMs, F4/80 cells were additionally MACS-enriched (using anti-CD11b microbeads) (Sigma).

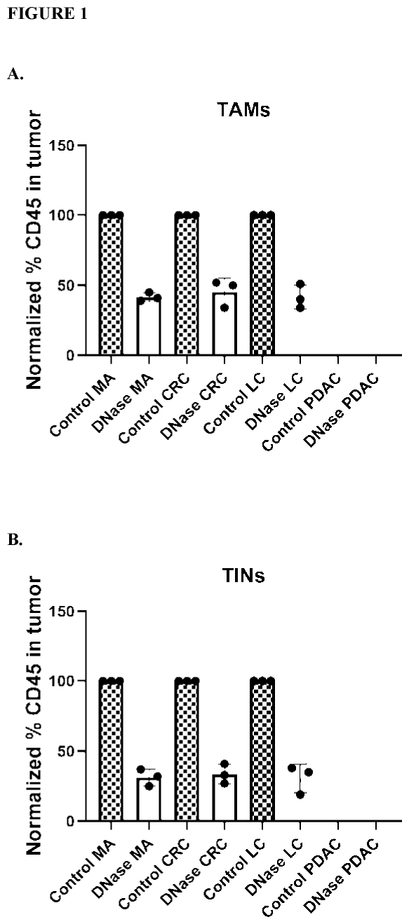

The amounts of TAMs and TINs following the treatment with DNase are shown in FIGS. 1A and 1B, respectively, as % to the relative controls (taken as 100%). The data clearly demonstrate that daily IV injections of DNase I enzyme provide statistically significant (p<0.05) reduction of TAMs and TINs.

### Example 2. Effect of Different Administration Regimens of Deoxyribonuclease Enzyme on TAMs and TINs Abundance within Tumor Tissue

1×10e5 Panc2 cells in 100 μl RPMI were injected subcutaneously into syngeneic C57Bl/6 female mice that were from six to eight weeks of age.

Six days post tumor implantation, animals were randomly assigned to groups (N=3):
Group 1: untreated control;
Group 2: IV injections of human recombinant DNase I enzyme (Kevelt AS; SEQ ID NO: 1) at 1 mg/kg dose daily for 7 days;
Group 3: IV injections of human recombinant DNase I enzyme (Kevelt AS; SEQ ID NO: 1) at 2 mg/kg dose, daily for 14 days;
Group 4: IV injections of human recombinant DNase I enzyme (Kevelt AS; SEQ ID NO: 1) at 2 mg/kg dose, daily for 7 days;
Group 5: IV injections of human recombinant DNase I enzyme (Kevelt AS; SEQ ID NO: 1) at 2 mg/kg dose, daily for 2 days;
Group 6: IV injections of human recombinant DNase I enzyme (Kevelt AS; SEQ ID NO: 1) at 2 mg/kg dose, for 2 days every week for three weeks;
Group 7: IV injections of human recombinant DNase I enzyme (Kevelt AS; SEQ ID NO: 1) at 2 mg/kg dose, for 2 days on weeks 1 and 3 for three weeks.

To isolate TAMs and TINs, tumors were extracted, and treated with 12 U/mL collagenase I, 450 U/mL collagenase IV, and 50 U/mL DNase I. Debris and dead cells were removed with density gradients. To purify TAMs, F4/80 cells were additionally MACS-enriched (using anti-CD11b microbeads).

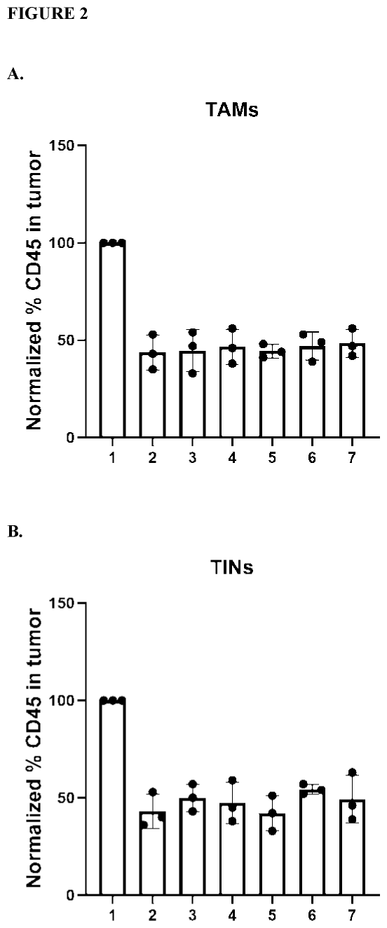

The amounts of TAMs and TINs are shown in FIGS. 2A and 2B, respectively.

Surprisingly, all of the used regimens of DNase I administration provided statistically significant (p<0.05) reduction of TAMs and TINs.

### Example 3. Effect of Different Time of Deoxyribonuclease Enzyme Therapy Initiation on TAMs and TINs Abundance within Tumor Tissue

3×10e6 of mammary adenocarcinoma cells in 100 μl RPMI were injected into syngeneic Balb/c female mice that were from six to eight weeks of age.

Animals were randomly assigned to groups (N=3):
Group 1: untreated control;
Group 2: IV injections of human recombinant DNase I enzyme (Kevelt AS; SEQ ID NO: 1) at 2 mg/kg dose every 3 days starting from day 2 post tumor implantation for 21 days total;
Group 3: IV injections of human recombinant DNase I enzyme (Kevelt AS; SEQ ID NO: 1) at 2 mg/kg dose every 3 days starting from day 6 post tumor implantation for 21 days total;
Group 4: IV injections of human recombinant DNase I enzyme (Kevelt AS; SEQ ID NO: 1) at 2 mg/kg dose every 3 days starting from day 10 post tumor implantation for 21 days total;
Group 5: IV injections of human recombinant DNase I enzyme (Kevelt AS; SEQ ID NO: 1) at 2 mg/kg dose every 3 days starting from day 14 post tumor implantation for 21 days total;
Group 6: IV injections of human recombinant DNase I enzyme (Kevelt AS; SEQ ID NO: 1) at 2 mg/kg dose every 3 days starting from day 18 post tumor implantation for 21 days total;

To isolate TAMs and TINs, tumors were extracted, and treated with 12 U/mL collagenase I, 450 U/mL collagenase IV, and 50 U/mL DNase I. Debris and dead cells were removed with density gradients. To purify TAMs, F4/80 cells were additionally MACS-enriched (using anti-CD11b microbeads).

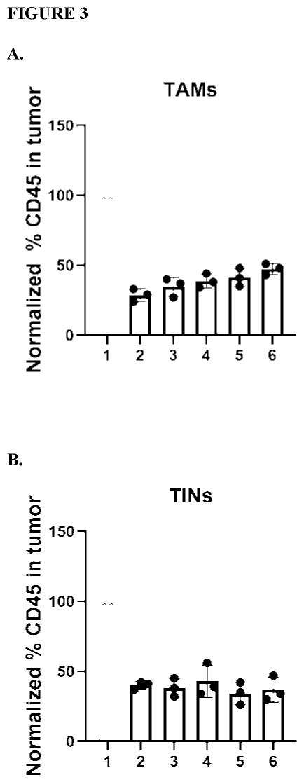

The amounts of TAMs and TINs are shown in FIGS. 3A and 3B, respectively.

Unexpectedly, the administration of DNase I even at late stages of tumor development provided statistically significant (p<0.05) reduction of TAMs and TINs.

### Example 4. Effect of Combining Deoxyribonuclease Enzyme Therapy with Immune Checkpoint Inhibitors on TAMs and TINs Abundance within Tumor Tissue

1×10e6 CT26 or MC38 cells in 100 μl RPMI were injected into female mice that were from six to eight weeks of age.

Treatment was started from day 3 or day 10 post tumor implantation.

Animals were randomly assigned to groups (N=3):
Group 1: untreated control;
Group 2: IV injections of human recombinant DNase I enzyme (Kevelt AS; SEQ ID NO: 1) at 2 mg/kg dose every 3 days starting from day 3 post tumor implantation;
Group 3: IV injections of human recombinant DNase I enzyme (Kevelt AS; SEQ ID NO: 1) at 2 mg/kg dose every 3 days starting from day 10 post tumor implantation for 21 days total;
Group 4: IV injections of anti-mouse CTLA4 antibody (BEO164, InVivoMAb, 200 μg per dose) every 3 days starting from day 3 post tumor implantation for 21 days total;
Group 5: IV injections of anti-mouse CTLA4 antibody (BEO164, InVivoMAb, 200 μg per dose) every 3 days starting from day 10 post tumor implantation for 21 days total;
Group 6: IV injections of anti-mouse CTLA4 antibody (BEO164, In VivoMAb, 200 μg per dose) every 3 days starting from day 3 post tumor implantation+IV injections of human recombinant DNase I enzyme (Kevelt AS; SEQ ID NO: 1) at 2 mg/kg dose every 3 days starting from day 3 post tumor implantation for 21 days total;
Group 7: IV injections of anti-mouse PD1 antibody (BEO146, In VivoMAb, 200 μg per dose) every 3 days starting from day 10 post tumor implantation+IV injections of human recombinant DNase I enzyme (Kevelt AS; SEQ ID NO: 1) at 2 mg/kg dose every 3 days starting from day 10 post tumor implantation for 21 days total;
Group 8: IV injections of anti-mouse PD1 antibody (BEO146, In VivoMAb, 200 μg per dose) every 3 days starting from day 3 post tumor implantation for 21 days total;
Group 9: IV injections of anti-mouse PD1 antibody (BEO146, In VivoMAb, 200 μg per dose) every 3 days starting from day 10 post tumor implantation for 21 days total;
Group 10: IV injections of anti-mouse PD1 antibody (BEO146, InVivoMAb, 200 μg per dose) every 3 days starting from day 3 post tumor implantation+IV injections of human recombinant DNase I enzyme (Kevelt AS; SEQ ID NO: 1) at 2 mg/kg dose every 3 days starting from day 3 post tumor implantation for 21 days total;
Group 11: IV injections of anti-mouse PD1 antibody (BEO146, InVivoMAb, 200 μg per dose) every 3 days starting from day 10 post tumor implantation+IV injections of human recombinant DNase I enzyme (Kevelt AS; SEQ ID NO: 1) at 2 mg/kg dose every 3 days starting from day 10 post tumor implantation for 21 days total;
Group 12: IV injections of anti-mouse OX-40 antibody (BE0031, In VivoMAb, 200 μg per dose) every 3 days starting from day 3 post tumor implantation for 21 days total;
Group 13: IV injections of anti-mouse OX-40 antibody (BE0031, InVivoMAb, 200 μg per dose) every 3 days starting from day 3 post tumor implantation+IV injections of human recombinant DNase I enzyme (Kevelt AS; SEQ ID NO: 1) at 2 mg/kg dose every 3 days starting from day 3 post tumor implantation for 21 days total;
Group 14: IV injections of anti-mouse OX-40 antibody (BE0031, InVivoMAb, 200 μg per dose) every 3 days starting from day 10 post tumor implantation for 21 days total;
Group 15: IV injections of anti-mouse OX-40 antibody (BE0031, InVivoMAb, 200 μg per dose) every 3 days starting from day 10 post tumor implantation+IV injections of human recombinant DNase I enzyme (Kevelt AS; SEQ ID NO: 1) at 2 mg/kg dose every 3 days starting from day 10 post tumor implantation for 21 days total.

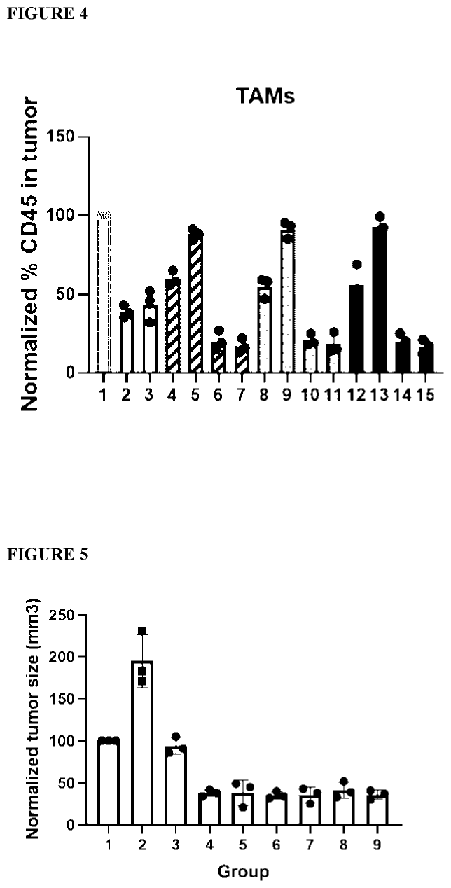

TAMs were extracted from tumors as described previously. The amounts of TAMs and TINs are shown in FIG. 4.

When used alone, checkpoint inhibitors statistically significantly decreased TAMs abundance at the tumor site at the early stage of tumor progression but failed to do so once they were used at a more advanced stage of the disease (when used in animals after 10 days post tumor implantation). Unexpectedly, when combined with DNase I, immune checkpoint inhibitors continued to significantly (p<0.05) reduce TAMs.

### Example 5. Effects of Different Types of DNase Enzymes on Tumor Microbiome

Fusobacterium nucleatum (F. nucleatum) is an oral anaerobe recently found to be prevalent in human colorectal cancer (CRC), breast cancer and some other cancers where it is associated with poor treatment outcome and tumor immunosuppressive environment. Inoculation with F. nucleatum leads to tumor colonization, suppresses accumulation of tumor infiltrating T cells and promotes tumor growth and metastatic progression. See, e.g., Kostic et al., Cell host & microbe, 2013, 14(2):207-15; Van der Merwe et al., Immunology Letters, 2021, 232:60-66.

F. nucleatum (ATCC 25586) were cultivated anaerobically in 5% CO₂ at 37° C. on Columbia agar supplemented with 5% sheep erythrocytes. Breast cancer cell line AT3 (ATCC) was injected into the mammary fat pad of C57BL/6 from six to eight weeks old. Animals were randomly assigned to groups (N=3). Day of study termination 24.
Group 1: untreated control;
Group 2: Injected with 7.5×10e7 F. nucleatum ATCC 23726;
Group 3: Injected with 7.5×10e7 F. nucleatum ATCC 23726 and on days 2, 3, 4, 5, 6, and 7 post tumor F. nucleatum implantation, animals were treated with metronidazole 10 mg/kg;
Group 4: Injected with 7.5×10e7 F. nucleatum ATCC 23726 and treated with human recombinant DNase I enzyme (Kevelt AS; SEQ ID NO: 1) at 2 mg/kg dose given every 3 days starting from day 1 post bacterial injection;
Group 5: Injected with 7.5×10e7 F. nucleatum ATCC 23726 and treated with mut DNase I enzyme (Kevelt AS) (SEQ ID NO: 2) at 2 mg/kg dose given every 3 days starting from day 1 post bacterial injection;
Group 6: Injected with 7.5×10e7 F. nucleatum ATCC 23726 and treated with DNase-1-like 3 (Human Microbiology Institute, NY, USA) (SEQ ID NO: 3) at 2 mg/kg dose given every 3 days starting from day 1 post bacterial injection;
Group 7: Injected with 7.5×10e7 F. nucleatum ATCC 23726 and treated with DNase-1-like 3 (SEQ ID NO: 3) at 2 mg/kg dose given on days 1 and 4 post bacterial injection;
Group 8: Injected with 7.5×10e7 F. nucleatum ATCC 23726 and treated with DNase-1-like 2 (Human Microbiology Institute, NY, USA) (SEQ ID NO: 4) at 2 mg/kg dose given every 3 days starting from day 1 post bacterial injection;
Group 9: Injected with 7.5×10e7 F. nucleatum ATCC 23726 and treated with DNase-1-like 1 (Kevelt AS) (SEQ ID NO: 5) at 2 mg/kg dose given every 3 days starting from day 1 post bacterial injection.

Tumor volume was measured with calipers and estimated using the ellipsoidal formula. The data are shown in FIG. 5. The data clearly show that IV injections of different types of human recombinant DNase I enzyme provide statistically significant (p<0.05) prevention of F. nucleatum triggered tumor growth and prevented F. nucleatum's pro-tumogenic effect.

On the day of study termination presence of F. nucleatum in the tumor tissue was confirmed by plating tumor homogenate of the nutrient medium, incubation at anerobic conditions at 37° C. for 48 hours. Additional PCR analysis of both tumor tissues and colonies that gave growth were conducted. Data are shown on the Table 1.

TABLE 1
Quantification of F. nucleatum at the tumor tissue

| | | Normalized* amount of F. |
|---|---|---|
| | | nucleatum within 1 g of |
| Group | F. nucleatum growth | tumor tissue |
| 1 | − | 0 |
| 2 | + | 100% |
| 3 | − | 0 |
| 4 | + | 99% ± 5.568 |
| 5 | + | 100% ± 12.767 |
| 6 | + | 107.3% ± 14.012 |
| 7 | + | 92.33% ± 3.786 |
| 8 | + | 98.33% ± 6.807 |
| 9 | + | 107% ± 25.515 |

*Amount of F. nucleatum was normalized as the percentage compared with the average F. nucleatum in the group 2 (positive control set as 100%) in each experiment. Data are shown as ± SD.

Data clearly show that DNase has not altered the amount of F. nucleatum at the tumor and does not possess antibacterial activity in these settings. Unexpectedly, DNase I significantly inhibited pro-tumorogenic effect of F. nucleatum. In this study, the regimen of DNase administration had little effect on the outcome and various types of DNase worked equally well.

### Example 6. Effects of Tumor Microbiome on Antitumor Activity of Immune Checkpoint Inhibitors

F. nucleatum ATCC 25586 were cultivated anaerobically in 5% CO₂ at 37° C. on Columbia agar supplemented with 5% sheep erythrocytes. Breast cancer cell line AT3 was injected into the mammary fat pad of C57BL/6 mice from six to eight weeks old with 18-22 g body weight. Animals were randomly assigned to groups (N=5). Study was terminated on day 21.
Group 1: untreated control;
Group 2: Injected with 7.5×10e7 F. nucleatum ATCC 23726;
Group 3: Injected with 7.5×10e7 F. nucleatum ATCC 23726 and on days 2, 3, 4, 5, 6, 7 post tumor F. nucleatum implantation, animals were treated with metronidazole 10 mg/kg (Sigma);
Group 4: Injected with 7.5×10e7 F. nucleatum ATCC 23726 and treated with human recombinant DNase I (Kevelt AS; SEQ ID NO: 1) at 2 mg/kg dose given every 3 days starting from day 1 post bacterial injection;
Group 5: Injected with anti-mouse CTLA4 antibody (BEO164, In VivoMAb, 200 μg per dose) every 2 days;
Group 6: Injected with 7.5×10e7 F. nucleatum ATCC 23726 and treated with anti-mouse CTLA4 antibody (BEO164, InVivoMAb, 200 μg per dose) given every 2 days starting from bacterial injection;
Group 7: Treated with human recombinant DNase I (Kevelt AS; SEQ ID NO: 1) at 2 mg/kg dose every 3 days starting from day 1 post bacterial injection and anti-mouse CTLA4 antibody (BEO164, In VivoMAb, 200 μg per dose) given every 2 days starting from bacterial injection; Group 8: Injected with 7.5×10e7 F. nucleatum ATCC 23726 and treated with human recombinant DNase I (Kevelt AS; SEQ ID NO: 1) at 2 mg/kg dose given every 3 days starting from day 1 post bacterial injection and anti-mouse CTLA4 antibody (BEO164, InVivoMAb, 200 μg per dose) given every 2 days starting from bacterial injection.

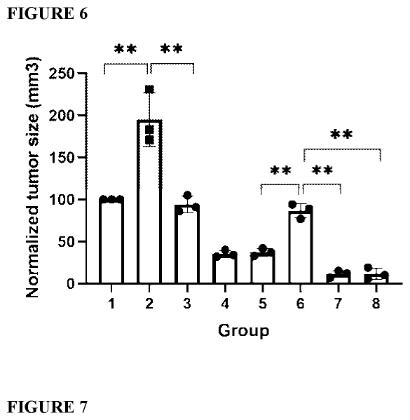

Tumor volume was measured with calipers and estimated using the ellipsoidal formula. The data are shown in FIG. 6.

On the last day, the presence of F. nucleatum in the tumor tissue was confirmed by plating tumor homogenate on the nutrient medium, incubating at anerobic conditions at 37° C. for 48 hours. Additional PCR analysis of both tumor tissues and colonies that gave growth was conducted. Data are shown on the Table 2.

TABLE 2
Quantification of F. nucleatum in the tumor tissue

| | | Normalized* amount of F. |
|---|---|---|
| | | nucelatum within 1 g of |
| Group | F. nucleatum growth | tumor tissue |
| 1 | − | 0 |
| 2 | + | 100% |
| 3 | − | 0 |
| 4 | − | 0 |
| 5 | − | 0 |
| 6 | + | 100.67% ± 13.503 |
| 7 | − | 0 |
| 8 | + | 108% ± 25.120 |

*Amount of F. nucleatum was normalized as the percentage compared with the average F. nucleatum in the group 2 (positive control set as 100%) in each experiment. Data re shown as ± SD.

The data clearly demonstrate that F. nucleatum significantly inhibited anticancer activity of immune checkpoint inhibitor anti-CTLA4 antibody. Unexpectedly, combined treatment with the checkpoint inhibitor and DNase I enzyme ameliorated pro-cancerogenic and anti-immunooncology activity of F. nucleatum (p<0.05), while not affecting the viability of this microorganism.

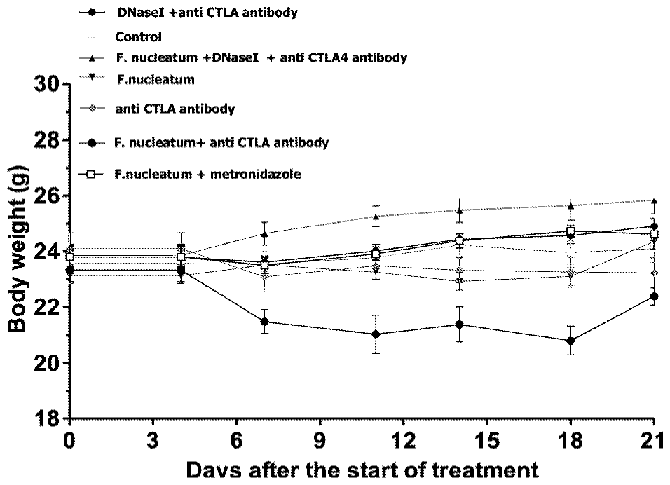

Another unexpected finding from this study was that F. nucleatum increased toxic effects of anti-CTLA4 antibody checkpoint inhibitor treatment and combined treatment with this checkpoint inhibitor and DNase I ameliorated F. nucleatum-induced toxicity as well as anti-CTLA4 antibody-induced toxicity. Specifically, as shown in FIG. 7, the administration of anti-CTLA4 antibody (at 3 mg/kg) or F. nucleatum (7.5×10e7) individually halted growth of body weight in treated animals, combination of anti-CTLA4 antibody and F. nucleatum lead to intensive weight loss, and DNase I treatment (at 2 mg/kg) prevented anti-CTLA4 antibody- and/or F. nucleatum-induced toxicity and resulted in weight gain.

Sequences

SEQ ID NO: 1-mature wild-type (WT) human DNase I (without secretory signal
sequence; Genbank Accession No. 4AWN_A):
LKIAAFNIQTFGETKMSNATLVSYIVQILSRYDIALVQEVRDSHLTAVGKLLDNLNQDAP
DTYHYVVSEPLGRNSYKERYLFVYRPDQVSAVDSYYYDDGCEPCGNDTFNREPAIVRFF
SRFTEVREFAIVPLHAAPGDAVAEIDALYDVYLDVQEKWGLEDVMLMGDFNAGCSYV
RPSQWSSIRLWTSPTFQWLIPDSADTTATPTHCAYDRIVVAGMLLRGAVVPDSALPFNF
QAAYGLSDQLAQAISDHYPVEVMLK
SEQ ID NO: 2-mature human DNAse I mutant (without secretory signal sequence);
the mutated residues as compared to SEQ ID NO: 1 are in bold and underlined:
LKIAAFNIRTFGRTKMSNATLVSYIVQILSRYDIALVQEVRDSHLTAVGKLLDNLNQDAP
DTYHYVVSEPLGRKSYKERYLFVYRPDQVSAVDSYYYDDGCEPCGNDTFNREPFIVRFF
SRFTEVREFAIVPLHAAPGDAVAEIDALYDVYLDVQEKWGLEDVMLMGDFNAGCSYV
RPSQWSSIRLWTSPTFQWLIPDSADTTATPTHCAYDRIVVAGMLLRGAVVPDSALPFNF
QAAYGLSDQLAQAISDHYPVEVMLK
SEQ ID NO: 3-human DNase-I-like 3
MSRELAPLLLLLLSIHSALAMRICSFNVRSFGESKQEDKNAMDVIVKVIKRCDIILVMEIK
DSNNRICPILMEKLNRNSRRGITYNYVISSRLGRNTYKEQYAFLYKEKLVSVKRSYHYH
DYQDGDADVFSREPFVVWFQSPHTAVKDFVIIPLHTTPETSVKEIDELVEVYTDVKHRW
KAENFIFMGDFNAGCSYVPKKAWKNIRLRTDPRFVWLIGDQEDTTVKKSTNCAYDRIV
LRGQEIVSSVVPKSNSVFDFQKAYKLTEEEALDVSDHFPVEFKLQSSRAFTNSKKSVTLR
KKTKSKRS
SEQ ID NO: 4-human DNase-I-like 2
MGGPRALLAALWALEAAGTAALRIGAFNIQSFGDSKVSDPACGSIIAKILAGYDLALVQ
EVRDPDLSAVSALMEQINSVSEHEYSFVSSQPLGRDQYKEMYLFVYRKDAVSVVDTYL
YPDPEDVFSREPFVVKFSAPGTGERAPPLPSRRALTPPPLPAAAQNLVLIPLHAAPHQAV
AEIDALYDVYLDVIDKWGTDDMLFLGDFNADCSYVRAQDWAAIRLRSSEVFKWLIPDS
ADTTVGNSDCAYDRIVACGARLRRSLKPQSATVHDFQEEFGLDQTQALAISDHFPVEVT
LKFHR
SEQ ID NO: 5-human DNase-I-like 1
MHYPTALLFLILANGAQAFRICAFNAQRLTLAKVAREQVMDTLVRILARCDIMVLQEVV
DSSGSAIPLLLRELNRFDGSGPYSTLSSPQLGRSTYMETYVYFYRSHKTQVLSSYVYNDE
DDVFAREPFVAQFSLPSNVLPSLVL VPLHTTPKAVEKELNALYDVFLEVSQHWQSKDVI
LLGDFNADCASLTKKRLDKLELRTEPGFHWVIADGEDTTVRASTHCTYDRVVLHGERC
RSLLHTAAAFDFPTSFQLTEEEALNISDHYPVEVELKLSQAHSVQPLSLTVLLLLSLLSPQ
LCPAA

### REFERENCES

Song, L. et al., “NLRP3 Inflammasome in Neurological Diseases, from Functions to Therapies” Front. Cell. Neurosci., 2017, Vol. 11, No. 63.
Fleischhacker M. et al., “Circulating nucleic acids (CNAs) and cancer: a survey” Biochim Biophys Acta, 2007, 1775(1): 181-232.
Demers, M. at al., “Cancers predispose neutrophils to release extracellular DNA traps that contribute to cancer-associated thrombosis” PNAS, 2012, 109(32): 13076-13081.
Garcia-Olmo D. C. and García-Olmo, D. “Biological role of cell-free nucleic acids in cancer: the theory of genometastasis” Crit Rev Oncolog., 2013, 18:153-161.
Sawyers 2008 C. L., “The cancer biomarker problem” Nature, 2008, 452(7187):548-552.
Butt A. N. et al. Overview of circulating nucleic acids in plasma/serum. Ann. N.Y. Acad. Sci., 2008, 1137:236-242.
Schwarzenbach H. et al., “Detection and monitoring of cell-free DNA in blood of patients with colorectal cancer” Ann. N.Y. Acad. Sci., 2008, 1137:190-196.
Zenaro E, et al., Neutrophils promote Alzheimer's disease-like pathology and cognitive decline via LFA-1 integrin. Nat Rev Nephrol., 2015, Author manuscript; available in PMC 2017 Jul. 14.
Fushi, W. et al., “Extracellular DNA in Pancreatic Cancer Promotes Cell Invasion and Metastasis” Cancer Res., 2013, 73:4256-4266.
Tohme, S. et al., “Neutrophil Extracellular Traps Promote the Development and Progression of Liver Metastases after Surgical Stress” Cancer Res., 2016, 76(6): 1367-1380.
Patutina, O. et al., Inhibition of metastasis development by daily administration of ultralow doses of RNase A and DNase I. Biochimie., 2011, 93(4): 689-96.
Dan Li, DNase I Treatment Reduces GVHD in Mice. Biology of Blood and Marrow Transplantation, Volume 21, Issue 2, Page S339.
Darvin, P. et al., “Immune checkpoint inhibitors: recent progress and potential biomarkers” Experimental & molecular medicine, 2018, 50(12): 1-1.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1.-3. (canceled)

4. A method of treating a cancer in a subject in need thereof, comprising administering to the subject an effective amount of a deoxyribonuclease (DNase) enzyme and a second immunomodulator.

5. The method of claim 4, wherein the administration of the DNase enzyme is effective to reduce the number and/or activity of the tumor associated macrophages (TAMs) and/or tumor infiltrating neutrophils (TINs) in the immunosuppressive tumor cell microenvironment.

6. The method of claim 1, wherein the second immunomodulator is an immune checkpoint modulator.

7. The method of claim 4, wherein the second immunomodulator is a modulator of an immune checkpoint module selected from PD-1, CD28, CTLA-4, CD137, CD40, CD134 (OX-40), ICOS, KIR, LAGS, CD27, TIM-3, BTLA, GITR, TCR, 4-1BB, TIGIT, CD96, CD226, KIR2DL, VISTA, HLLA2, TLIA, DNAM-1, CEACAM1, CD155, IDO, TGF-beta, IL-10, IL-2, IL-15, CSF-1, IL-6, adenosine A2A receptor (A2AR), and a ligand thereof.

8. The method of claim 6, wherein the immune checkpoint modulator is an immune checkpoint inhibitor.

9. The method of claim 8, wherein the immune checkpoint inhibitor is an antibody that specifically binds CTLA-4, PD-1, OX-40, PD-L1, or PD-L2.

10. The method of claim 8, wherein the immune checkpoint inhibitor is an antibody that specifically binds to CTLA-4, PD-1, PD-L1, PD-L2, A2AR, B7-H3, B7-H4, BTLA, IDO, KIR, LAG 3, NOX2, TIM-3, VISTA, or SIGLEC7.

11. The method of claim 8, wherein the immune checkpoint inhibitor is selected from ipilimumab, tremelimumab, nivolumab, pembrolizumab, pidilizumab, MEDI0680, atezolizumab, avelumab, durvalumab, cemiplimab, and any combinations thereof.

12. The method of claim 8, wherein the immune checkpoint inhibitor is pembrolizumab.

13. The method of claim 4, wherein the DNase enzyme is selected from human DNase I, human DNase-I-like 3 (D1L3), human DNase-I-like 2 (D1L2), human DNase-I-like 1 (D1L1), DNase X, DNase γ, DNase II, DNase IIα, DNase IIβ, and Caspase-activated DNase (CAD).

14.-117. (canceled)
